(19) United States
(12) Patent Application Publication
(10) Pub. No.: US 2026/0227297 A1
(43) Pub. Date: Aug. 6, 2026

(54) CROSSLINKERS FOR EXPANSION MICROSCOPY

(71) Applicant: Wisconsin Alumni Research Foundation, Madison, WI (US)

(72) Inventor: Aussie Suzuki, Madison, WI (US)

(21) Appl. No.: 19/043,198

(22) Filed: Jan. 31, 2025

(51) Int. Cl. G01N 1/36; G01N 33/50

(52) U.S. Cl. CPC G01N 1/36; G01N 33/50; G01N 2001/366

(57) ABSTRACT

Disclosed herein are kits and methods for modified expansion microscopy, including a mold having a sealing surface configured to form an airtight seal with a sealing material to exclude air bubbles from the mold, a crosslinking agent configured to covalently bind to both a biological element of a sample and to an expansion gel, and instructions for use of the mold in an expansion microscopy method. The instructions include a description of an expansion microscopy method that includes at least one method step that involves gelling in the absence of oxygen, and a written or visual representation of execution of the expansion microscopy method by conducting the at least one method step that involves gelling in the mold by forming an airtight seal against the sealing surface with a sealing material to exclude visible air bubbles from the mold upon sealing.

### STATEMENT REGARDING FEDERALLY SPONSORED RESEARCH

This invention was made with government support under GM147525 awarded by the National Institutes of Health. The government has certain rights in the invention.

### FIELD OF THE INVENTION

The disclosed technology is generally directed to super-resolution light microscopy. More particularly the technology is directed to crosslinkers used in expansion microscopy.

### BACKGROUND OF THE INVENTION

Light microscopy is one of the powerful tools to study protein dynamics and protein functions in both fixed and living cells. The diffraction limit (optical resolution limit) in light microscopy is ~250 nm in x, y-axis, and ~600 nm in z-axis without any mathematical image processing. Since many of cellular system and protein architectures in cells have less than diffraction limited size, it is a big challenge to study those functions using light microscopy. To overcome this technical difficulty, researchers mainly use two approaches; one is to use electron microscopy, and another is to use super-resolution light microscopy. Advantage of the use of electron microscopy can achieve ~1 nm resolution without any image processing, but it has multiple disadvantages, including high technical difficulty in preparing samples, the method only works with fixed samples, and difficulties in labeling a target protein, especially with multiple targets. Because of these reasons, developing super-resolution light microscopy techniques is highly demanded. Recent technology allows to reach ~50 to ~150 nm resolution using super-resolution microscopes, but it requires mathematical image processing, very high signal to noise ratio images, to use with specific dyes, and the use of very expensive equipment, that make existing methods difficult to use in broad research fields and researchers. Additionally, majority of super-resolution microscopy require mathematical image post-image processing to obtain better resolution, it is always needed to consider whether the image processed by super-resolution microscopy is real or not. There is strong motivation in the field to develop new instrument to achieve better resolution, but recent studies proposed an alternative solution: making samples larger rather than developing new expensive microscopes to reach better resolution.

This alternative solution is called expansion microscopy and allows expansion of specimens (cells, human organoids, tissues, etc.) with regular staining (multiple colors) and can image with any light microscopes, including existing super-resolution microscopes. The basic concept of expansion microscopy is to imbed conventionally fluorophore-labeled biological samples in a polymer gel, bond the gel to the fluorophore and/or a structure of the biological sample, and expand the gel—and consequently, expand the biological sample along with it—to achieve a sample that is physically larger in volume. As a general overview, expansion microscopy includes the steps of (1) labeling a feature of a biological sample with an imaging probe, (2) anchoring the imaging probe to a component of the expansion gel using a crosslinking molecule, (3) polymerization of a gelation solution of monomers cast across the biological sample to form a swellable hydrogel, integrating the anchored targets within the biological sample, (4) homogenization of the biological sample to disrupt the mechanical structure of the biological sample to allow for uniform expansion, (5) expansion of the gel, and biological sample with it, several fold, and (6) imaging the sample using fluorescence microscopy.

While there are several existing protocols, their reproducibility and robustness are not well established. For example, the original protocol of expansion microscopy, despite being named “4× Expansion Microscopy,” had limited reproducibility in our and others' labs, and the purported 4× expansion was not achieved. Instead, expansions of about 2.5× were attained. Additionally, these existing methods required use of expensive equipment and have issues in 3D imaging. A newer protocol of expansion microscopy emerged, so-called “12× Expansion Microscopy,” but again, reproduction in our lab was not achievable and expansions of only 5.5× were achieved by this method. Accordingly, a need exists for improved techniques and materials for improving the performance of expansion microscopy methods to reliably produce 4-fold and larger expansion in range of sample types. Additionally, there is a need to improve the methods and materials used in anchoring a biological sample to the expansion gel for expansion microscopy.

A commonly used crosslinking molecule in expansion microscopy is acryloyl-X (AcX), which supports up to 4-fold expansion. However, it exhibits anisotropic expansion when exceeding this threshold. In contrast, glutaraldehyde (GA) can facilitate both 4-fold and 12-fold expansion and has the potential to enable even larger expansions. Despite their utility, both AcX and GA are associated with significant fluorescence signal loss from labeled dyes and challenges in expanding certain cellular components, including RNA, DNA, and specific tissue types. Consequently, there is a need in the field for development of additional crosslinking molecules and methods of their use to address these technical limitations.

### BRIEF SUMMARY OF THE INVENTION

Disclosed herein are kits and methods for anchoring a biological sample to an expansion gel for expansion microscopy.

Disclosed herein are kits for modified expansion microscopy (mExM), including a mold including a sealing surface configured to form an airtight seal with a sealing material to exclude air bubbles from the mold. The kits also include a crosslinking agent configured to covalently bind to both a biological element of a sample and to an expansion gel, and instructions for use of the mold in an expansion microscopy method. The instructions include a description of an expansion microscopy method that includes at least one method step that involves gelling in the absence of oxygen, and a written or visual representation of execution of the expansion microscopy method by conducting the at least one method step that involves gelling in the mold by forming an airtight seal against the sealing surface with a sealing material to exclude visible air bubbles from the mold upon sealing.

Also disclosed herein are methods of modified expansion microscopy (mExM). The methods include a) fixing a biological sample having a biological element to an inert surface, optionally locating the inert surface within a mold including a surface configured to form an airtight seal; b) exposing the biological sample to a crosslinking agent to produce a covalent bond between the crosslinking agent and the biological element; c) exposing the biological sample to a plurality of expansion monomers and polymerizing at least a portion of the expansion monomers to form covalent bonds between the crosslinking agent and the expansion monomers to form polymerized expansion monomers, d) optionally introducing the inert surface into the mold; e) introducing a gelling solution into the mold including the inert surface and the biological sample of interest; f) subsequent to step e), sealing the mold with a sealing material to exclude air bubbles from the mold; g) gelling the polymerized expansion monomers to form a gelled biological sample, the gelling proceeding without active deoxygenation; h) digesting the gelled biological sample by exposing the gelled biological sample to a digestion composition; i) expanding the gelled biological sample to form an expanded biological sample; and j) preparing the expanded biological sample for imaging.

The kits and methods disclosed herein include crosslinking agents, including glycidyl methacrylate (GMA), (2,5-dioxopyrrolidin-1-yl) 3-[(2,5,9-trimethyl-7-oxofuro[3,2-g]chromen-3-yl)methylsulfanyl]propanoate (AMT-NHS), glyoxal, acryloyl-X (AcX), glutaraldehyde (GA), or a combination thereof.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 is a schematic of components of kits, as disclosed herein.

FIG. 2 is a flowchart illustrating a method, in accordance with an aspect of the present disclosure.

FIG. 3 is a flowchart illustrating a method, in accordance with an aspect of the present disclosure.

FIG. 4A shows a schematic of a silicone mold, with a sealing surface.

FIG. 4B shows a schematic of a mold containing an expanded gel prepared for imaging.

### DETAILED DESCRIPTION OF THE INVENTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices and methods relating to modifying biological molecules are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

Kits and methods for improved cellular microscopy are disclosed. Specifically, improvements to expanded microscopy techniques are disclosed. Before describing specific kits in detail, it should be appreciated that the individual kits that are described can include components or features of the other described kits. One such combined kit includes all of the components and all of the instructions described in all of the kits. Each of the separately-described kits articulates a feature that achieves a particularly impressive results or that provides a particularly impressive simplification of existing techniques.

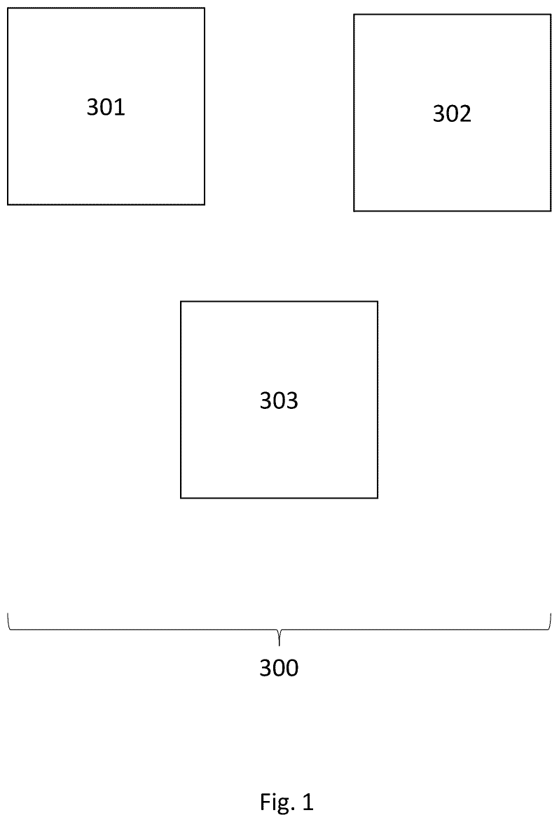

Referring to FIG. 1, a first kit (300) is disclosed that includes a mold (301), a crosslinking agent (302), and instructions (303) for use of the mold with expansion gels used in expansion microscopy on samples of interest. In some cases, samples of interest include cells, tissue culture cells, tissues, tissue samples including multiple cells, organoids, organs, embryos, and any combinations thereof.

In some cases, the mold can have a substantially cylindrical interior shape. The mold can have an opening, including a sealing surface. The mold includes a sealing surface configured to form an airtight seal with a sealing material. The airtight seal may be a temporary seal. The airtight seal may be made between the sealing surface and the sealing material excludes air bubbles from the mold. The airtight seal excludes air bubbles from the mold and avoids allowing air to contact the contents of the mold. The airtight seal is capable of being formed with manual force. The sealing material may comprise a hydrophobic film. In some cases, the sealing material comprises a wax material, a film, a nylon material, a silicone, a flexible hydrophobic material, or a combination thereof. In some cases, the sealing material comprises parafilm.

In some cases, the mold may be chemically and/or biochemically inert in that the mold may not chemically and/or biochemically reactive with or interfere with the sample of interest or the chemistry of the crosslinking agent or expansion gels used therein. In some cases, an inert surface can be positioned within the mold. In other cases, the inert surface can be outside of the mold and adapted to be receivable within the mold. Suitable materials that can provide an air- and/or water-tight seal when properly sealed as described herein, including hydrophobic materials, and combinations thereof. The materials are not necessarily rigid materials. The mold and/or inert surface may be comprised of a chemically and/or biochemically inert material such as polymeric materials (e.g., silicone, polystyrene, gum, etc.), crystalline materials (e.g., diamond) glasses (e.g., silicon dioxide), surfaces treated (e.g., functionalized or passivated) with chemically and/or biochemically inert functional groups (e.g., pegylated surface, gold surfaces treated with bovine serum albumin, thin oxide layer) to form one or more protective layers to inhibit interactions or reactions between the mold and/or inert surface and the contents contained therein, or any combinations thereof.

The mold may be dimensioned to receive a sample of interest. In some cases, the mold may be dimensioned to contain a sample which will undergo at least a 12-times increase in sample volume of the sample of interest.

The instructions include a description of an expanded microscopy (ExM) method and a written or visual representation of execution of the ExM method. The ExM method includes at least one method step that involves polymerization (e.g., gelling) in the absence of oxygen. The written or visual representation includes execution of the ExM method by conducting the at least one method step that involves polymerization in the mold by forming a hand-made seal with a sealing material and excluding visible oxygen bubbles (and avoid contacting air) from the mold upon sealing. Under established methods, active deoxygenation has been traditionally required, so this simplification of this process saves significant cost and effort.

The crosslinking agent is configured to covalently bind to both a biological element of a sample and to an expansion gel. In some cases, the crosslinking agent may include glycidyl methacrylate (GMA), (2,5-dioxopyrrolidin-1-yl)3-[(2,5,9-trimethyl-7-oxofuro[3,2-g]chromen-3yl)methylsulfanyl]propanoate (AMT-NHS), glyoxal, acryloyl-X (AcX), glutaraldehyde (GA), or a combination thereof. In some cases, multiple crosslinking agents may be used to target different biological elements within the same sample. The crosslinking agents disclosed herein may be used in the kits and methods disclosed in U.S. Patent Application Publication No. 2022/0074829 A1, which is incorporated by reference, in its entirety and for any purpose, herein.

As used herein, a “biological element” refers to a structure or feature of the sample of interest. When crosslinked to an expansion gel via a crosslinking agent, the biological elements are the biological structure which is expanding with the expansion of the expansion gel. In some cases, the biological element may include one or more of the following: nucleotides, proteins, organelles, single stranded DNA, double stranded DNA, single stranded RNA, double stranded RNA, DNA-RNA hybrids, higher order DNA/RNA structures or any combinations thereof.

A second kit is disclosed that includes a protein digestion buffer and instructions for use of the protein digestion buffer in an ExM method. The instructions include a description of an ExM method that includes at least one method step that involves a digestion step utilizing a protein digestion agent. The instructions further include a written or visual representation of execution of the ExM method by utilizing the protein digestion buffer in the digestion step. The written or visual representation including reaction conditions for the digestion step.

A third kit is disclosed that includes the protein digestion buffer and the mold. The protein digestion buffer includes sodium dodecyl sulfate. The protein digestion agent is present in concentrations suitable for direct addition to the mold to affect a digestion step of an ExM method. The third kit can optionally include, but does not require, instructions.

The instructions for any of the first, second, or third kit can include any of the method steps, reaction conditions, or other details described below with respect to method 100, method 200, or the examples.

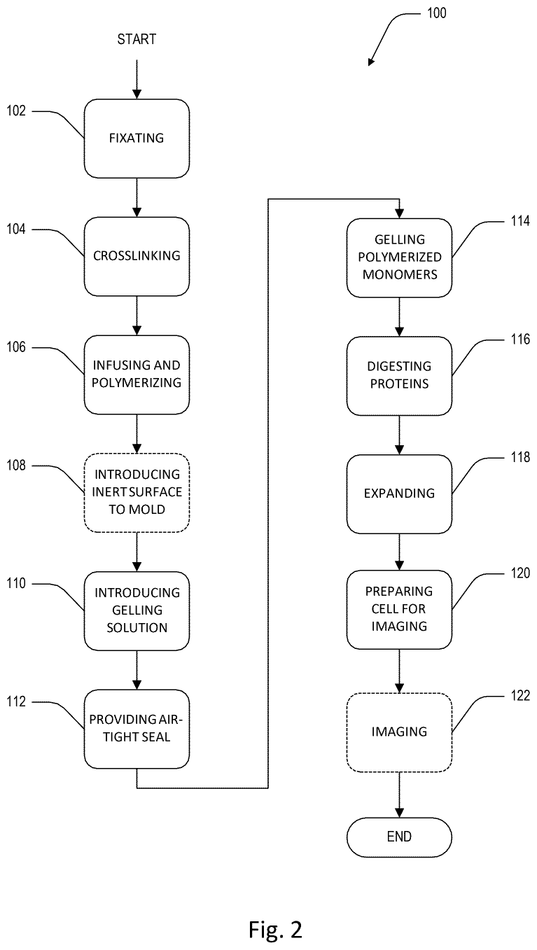

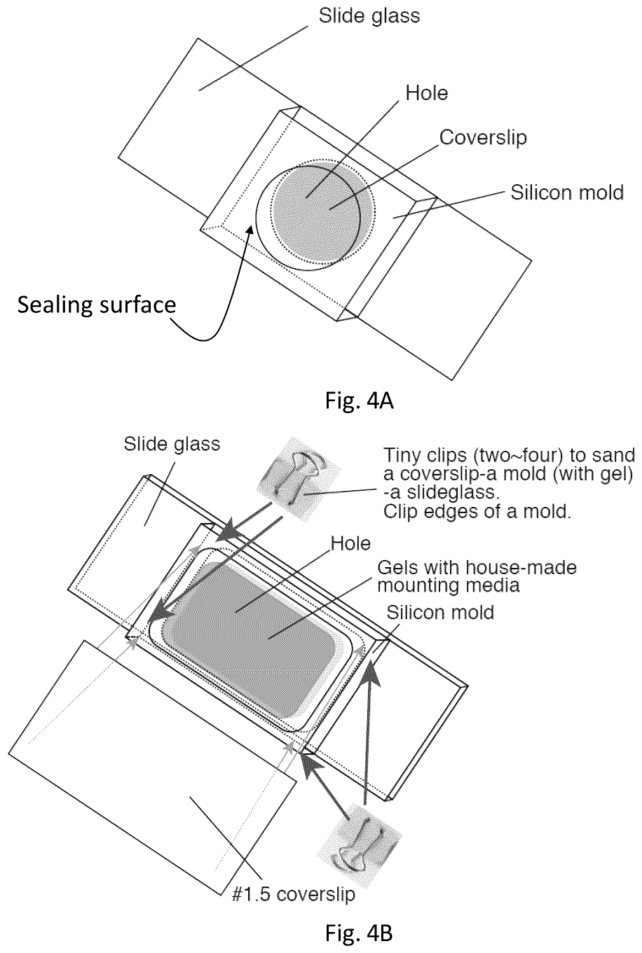

Referring to FIG. 2, a method 100 of ExM is disclosed. At process block 102, the method 100 includes fixating a sample of interest to an inert surface. In some cases, the inert surface can be positioned within a mold. In other cases, the inert surface can be outside of the mold and adapted to be receivable within the mold. At process block 104, the method 100 includes crosslinking biological elements of the sample of interest. In some cases, the crosslinking agent may include glycidyl methacrylate (GMA), (2,5-dioxopyrrolidin-1-yl)3-[(2,5,9-trimethyl-7-oxofuro[3,2-g]chromen-3yl)methylsulfanyl]propanoate (AMT-NHS), glyoxal, acryloyl-X (AcX), glutaraldehyde (GA), or a combination thereof. In some cases, multiple crosslinking agents may be used to target different biological elements within the same sample. Combinations of crosslinking agents may be used in sequential crosslinking steps or in a concurrent (e.g., one-pot) crosslinking step. At process block 106, the method 100 includes infusing the sample of interest with a plurality of ExM monomers and polymerizing at least a portion of the ExM monomers. As discussed elsewhere herein and specifically relevant to method 100, some polymerized ExM monomers do not properly gel in the presence of oxygen (i.e., those used to achieve a 12-fold expansion—monomers used for the 4-fold expansion can polymerize in the presence of oxygen). At optional process block 108, the method 100 optionally includes introducing the inert surface into the mold, as shown in FIG. 4A. Optional process block 108 is utilized in cases where the inert surface is positioned outside of the mold. At process block 110, the method 100 includes introducing a gelling solution into the mold including the inert surface and the sample of interest. At process block 112, the method 100 includes, subsequent to process block 110, providing an airtight seal to the mold. The airtight seal excludes air bubbles from the mold and avoids allowing air to contact the contents of the mold. The airtight seal is capable of being formed with manual force. At process block 114, the method 100 includes gelling the polymerized ExM monomers. The gelling proceeds without active deoxygenation. At process block 116, the method 100 includes digesting proteins within the sample of interest. At process block 118, the method 100 includes expanding the sample of interest by initiating expansion of the gelled, polymerized ExM monomers. At process block 120, the method 100 includes preparing the expanded sample of interest for imaging, for example as shown in FIG. 4B. At optional process block 122, the method 100 optionally includes imaging the prepared, expanded sample of interest.

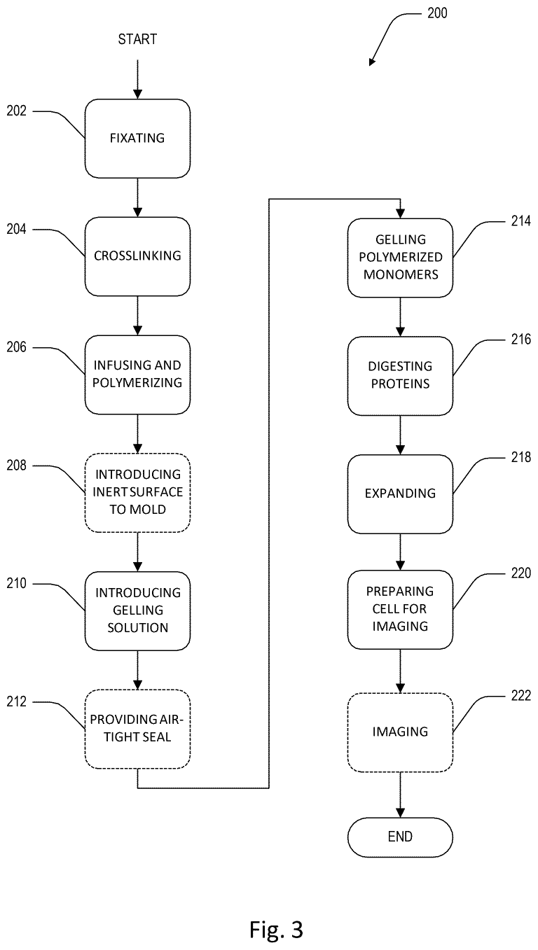

Referring to FIG. 3, a method 200 of ExM is disclosed. At process block 202, the method 200 includes fixating a sample of interest to an inert surface. In some cases, the inert surface can be positioned within a mold, as shown in FIG. 4A. In other cases, the inert surface can be outside of the mold and adapted to be receivable within the mold. At process block 204, the method 200 includes crosslinking portions of biological elements of the sample of interest. In some cases, the crosslinking agent may include glycidyl methacrylate (GMA), (2,5-dioxopyrrolidin-1-yl)3-[(2,5,9-trimethyl-7-oxofuro[3,2-g]chromen-3yl)methylsulfanyl]propanoate (AMT-NHS), glyoxal, acryloyl-X (AcX), glutaraldehyde (GA), or a combination thereof. In some cases, multiple crosslinking agents may be used to target different biological elements within the same sample. Combinations of crosslinking agents may be used in sequential crosslinking steps or in a concurrent (e.g., one-pot) crosslinking step. At process block 206, the method 200 includes infusing the sample of interest with a plurality of ExM monomers and polymerizing at least a portion of the ExM monomers. At optional process block 208, the method 200 optionally includes introducing the inert surface into the mold. Optional process block 208 is utilized in cases where the inert surface is positioned outside of the mold. At process block 210, the method 200 includes introducing a gelling solution into the mold including the inert surface and the sample of interest. At optional process block 212, the method 200 optionally includes providing an airtight seal to the mold. The airtight seal excludes air bubbles from the mold. The airtight seal excludes air bubbles from the mold. The airtight seal is capable of being formed with manual force. At process block 214, the method 200 includes gelling the polymerized ExM monomers. The gelling optionally proceeds without active deoxygenation. At process block 216, the method 200 includes digesting proteins within the sample of interest. The digesting comprises use of a protein digestion composition including a protein digestion agent and a protein digestion buffer. The protein digestion buffer includes sodium dodecyl sulfate. At process block 218, the method 200 includes expanding the sample of interest by expansion of the gelled, polymerized ExM monomers. At process block 220, the method 200 includes preparing the expanded sample of interest for imaging as, for example, in FIG. 4B. At optional process block 222, the method 200 optionally includes imaging the prepared, expanded sample of interest.

The first kit and the method 100 expressly require the use of the mold and the second kit, the third kit, and the method 200 optionally include it. For the avoidance of doubt, all features described herein with respect to the mold are contemplated for use with the first, second, and third kit and the methods 100, 200 described herein.

The methods 100, 200 involve use of an inert surface and the first, second, and third kit can optionally include it.

In some cases, the inert surface is positioned in the bottom of the mold in a permanent or semi-permanent fashion.

In some cases, the inert surface is adapted to be removed from and introduce to the mold. In some cases, the inert surface has a shape that is a cross-sectional shape of the mold. In some cases, particularly when the mold has a cylindrical shape, the inert surface is circular.

The inert surface can be glass. In some cases, the inert surface is a glass cover slip. In some cases, the glass cover slip is cut to a shape that is adapted for introduction into the mold.

The fixating of process blocks 102 and 202 can be standard biological sample fixating understood by those having ordinary skill in arts that involve the imaging of biological samples. In some cases, the fixating involves sub-steps including a sample preparation sub-step, a primary fixation sub-step, and a pre-ExM staining sub-step. The sample preparation sub-step can involve spreading a sample or interest or multiple samples of interest on the inert surface and growing the sample(s) of interest for at least 36 hours or at least 48 hours. The primary fixation sub-step can involve fixing the sample by introducing pre-warmed 3% PFA in PHEM buffer for 15 minutes at 37° C. Other fixation approaches (glutaraldehyde, methanol, acetone, and others) can be utilized, as will be appreciated by those having ordinary skill in the art. The inventors surprisingly discovered that using PFA in a phosphate buffered saline buffer system, or fixations with methanol or acetone showed less preservation effects compared to the use with PHEM or HEPES based buffer. Thus, in some cases, it can be preferable to exclude phosphate-buffered saline buffers and/or their components from the fixating step of methods 100, 200. The pre-ExM staining sub-step can involve a conventional immunostaining protocol, as would be understood by those having ordinary skill in the art. Conventional immunostaining can be achieved with Alexa Fluor™ 488, Alexa Fluor™ 568, Rhodamine Red-X, or other conventional dyes known to those in the art.

One non-limiting example of a pre-ExM staining sub-step process includes: 1) after samples are fixed, samples can be washed by a buffer, such as PHEM or PBS, one, two, or three times-if high concentrations of PFA or glutaraldehyde are utilized, a quenching step can be performed here with glycine or sodium borohydride containing PHEM or PBS; 2) blocking by bovine serum albumin (BSA), skim milk, or immunoglobulin G (IgG) for 15-30 minutes at room temperature; 3) incubation with primary antibodies in blocking buffer at 37° C. for 1 hour or 4° C. overnight; 4) washing samples with blocking buffer at least three times; 5) incubating with secondary antibodies in blocking buffer at 37° C. for 1 hour or 4° C. overnight; 6) washing samples with a buffer, such as PHEM or PBS; 7) DNA staining by a DNA dye, such as DAPI.

The crosslinking of process blocks 104 and 204 can be achieved with a crosslinking agent. The first, second, and third kit can also include the crosslinking agent. The crosslinking agent can include glycidyl methacrylate (GMA), (2,5-dioxopyrrolidin-1-yl)3-[(2,5,9-trimethyl-7-oxofuro[3,2-g]chromen-3-yl)methylsulfanyl]propanoate (AMT-NHS), glyoxal, acryloyl-X (AcX), glutaraldehyde (GA), or a combination thereof.

In some cases, glycidyl methacrylate (GMA) is used in a concentration from 0.008% to 1.0%, from 0.1% to 1.0%, from 0.008% to 0.75%, from 0.01% to 0.6%, from about 0.01% to 0.5%, by weight. In some cases, the GMA may be supplied in a buffer solution. In some cases, the sample may be incubated in a solution containing GMA for about 8 hours, about 9 hours, about 10 hours, about 11 hours, about 12 hours, about 13 hours, about 14 hours, about 15 hours, about 16 hours, about 17 hours, or about 18 hours. In some cases, GMA is used in conjunction with glutaraldehyde (GA) at the concentrations given above.

In some cases, (2,5-dioxopyrrolidin-1-yl)3-[(2,5,9-trimethyl-7-oxofuro[3,2-g]chromen-3-yl)methylsulfanyl]propanoate (AMT-NHS) is used in a concentration from 0.0075 mM to 2.0 mM, from 0.0075 to 1.5 mM, from 0.0095 mM to 1.25 mM, from 0.01 mM to 1.15 mM, or about 0.01 mM to 1.0 mM. In some cases, the AMT-NHS may be supplied in a buffer solution. In some cases the buffer solution is a physiological buffer solution, such as PBS, HEPES, or similar. In some cases, the sample may be incubated in a solution containing AMT-NHS for about 1 minute, about 5 minutes, about 10 minutes, about 20 minutes, about 30 minutes, about 45 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, or about 18 hours. In some cases, AMT-NHS is used in conjunction with glutaraldehyde (GA) at the concentrations given above.

In some cases, glyoxal is used in a concentration from 0.1% to 50%, from 0.25% to 20%, or from 1.0% to 10% by weight in a physiological buffer solution, such as PBS, HEPES, or similar. In some cases, glyoxal may be formulated at any of the above concentrations in a solution of acetic acid. In some cases, the acetic acid solution may contain from 0.1% to 50%, from 0.25% to 20%, or from 1.0% to 10% by weight acetic acid. In some cases, the sample may be incubated in a solution containing glyoxal for about 1 minute, about 5 minutes, about 10 minutes, about 20 minutes, about 30 minutes, about 45 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, or about 18 hours. In some cases, glyoxal is used in conjunction with glutaraldehyde (GA) at the concentrations given above.

In some cases, acryloyl-X (AcX) is used. Conventional ExM methods utilized AcX in higher concentrations than the present methods. Inventors surprisingly discovered that lower concentrations of AcX produced more reliable results than the higher concentrations used in conventional ExM methods (in fact, without wishing to be bound by any particular theory, the inventors believe that the higher concentration of AcX used in conventional ExM methods limit expansion). In conventional methods, the final concentration of AcX in the crosslinking step is ~0.1 mg/ml. In methods 100 and 200, where AcX is used without GA, the final concentration of AcX in this step can be between 0.001 and 0.05 mg/ml or between 0.01 and 0.05 mg/ml. The AcX can be provided in a buffer solution, such as a PBS buffer. In some cases, AcX and GA are used together in the concentrations described above.

In some cases, GA is used without another crosslinking agent. In such cases, the GA is used at a final concentration of between 0.1% and 2% by weight in a buffer solution, such as a PBS buffer.

For each of the crosslinking agents utilized, the crosslinking of process blocks 104 and 204 can involve incubation at room temperature with protection from light. The incubation can be for a length of time of between 5 minutes and 24 hours.

In some cases, the methods 100, 200 can include a washing step following the crosslinking of process blocks 104 and 204. This washing can be by a buffer, such as a PBS buffer, and can be repeated two, three or more times.

If the crosslinking agent includes GA, the methods 100, 200 can include an optional additional crosslinking by a 0.01-2% by weight GA in a buffer solution. The additional crosslinking can be done for between 5 minutes and 30 minutes or between 10 and 20 minutes at room temperature.

The gelling solution of methods 100, 200 of any of its components can be included in the first, second, or third kit.

The gelling solution includes ExM monomers. The degree of expansion is strongly linked to the ExM monomer that is chosen. For cases where ~x4 expansion is desired, the ExM monomers can include acrylamide, N,N′-methylenebisacrylamide, or a combination thereof. For cases where 10× expansion is desired, the ExM monomers can include N,N′-dimethylacrylamide (DMAA).

For 4-fold gels, three buffers can be used together. (1) 1×PBS, 2M NaCl, 2.5% w/v Acrylamide, 0.15% w/v N,N′-methylenebisacrylamide, 8.6% Sodium Acylate, (2) 10% Ammonium Persulfate, and (3) 10% w/v TEMED. All three solutions can be stored at −20° C. for few months. For gel polymerization, 95% of solution (1), 0.2% of both solution (2) and (3), and 0.1% of water are mixed. For 12-fold gels, following two buffers can be used together. (1) the molar ratio of DMAA (N,N′-dimethylacrylamide) to sodium acrylate is 4:1. For example, we typically make DMAA 1.335 g, Sodium, acrylate 0.32 g, and water 2.85 g in which total volume becomes ~4.5 ml. The solution can be stored at −20° C. for up to three weeks but no longer recommended. (2) 3.6% of KPS solution (in water). For gel polymerization, 10% of solution (1), 10% of solution (2), and ~0.4% of TEMED are mixed.

The introducing of the gelling solution of process blocks 110 and 210 can involve optionally washing the sample(s) of interest prior to actually introducing the gelling solution. In some cases, the introducing of process blocks 110 and 210 can include adding a buffer solution that does not include the ExM monomers to the mold and subsequently adding the ExM monomers (in most cases, in a buffer themselves).

Some conventional ExM methods that utilize DMAA require active deoxygenation. The inventors surprisingly discovered an elegant and inexpensive alternative to active deoxygenation. This alternative is the providing an airtight seal and excluding air bubbles of process blocks 112 or 212.

Gelling the polymerized ExM monomers of process blocks 114 and 214 can be achieved without active deoxygenation. Gelling the polymerized ExM monomers of process blocks 114 and 214 can involve incubation at room temperature plus or minus 5° C. for a length of time of between 1 hour and 6 hours, between 1.5 hours and 3 hours, or between 2 hours and 2.5 hours.

In one specific case, the providing an airtight seal and excluding air bubbles involves contacting the opening of the mold with a paraffin wax film, squeezing any excess air out of the mold, and then finally sealing the opening of the mold with the film.

The digesting of process blocks 116 and 216 can use a protein digestion composition. The protein digestion composition includes a protein digestion agent and a protein digestion buffer. The first, second, and third kit can include the protein digestion agent. The first, second, and third kit can include the protein digestion buffer.

The protein digestion agent can be a serin protease, such as Proteinase K, or a protein digestion enzyme understood by those having ordinary skill in the art to have digestion capabilities that are necessary for allowing the methods described herein to achieve their full expansions.

The protein digestion buffer includes sodium dodecyl sulfate (SDS) and a buffering component. The buffering component can include standard buffers known to those having ordinary skill in the art, such as TAE buffer (Tris, acetic acid, and EDTA). The protein digestion buffer can include SDS in an amount such that a final concentration in the protein digestion buffer is between 0.1% and 1%, including between 0.25% and 0.75%, or between 0.4% and 0.6%, where these percentages can be weight by volume or weight by weight. The protein digestion buffer is stable when stored at −20° C. for at least three months. In some cases, the protein digestion buffer includes 1× TAE (40 mM Trix, 20 mM acetic acid, 1 mM EDTA), 0.5% v/v of Triton-X, 0.1-1% of SDS, and 8~70 U of Proteinase K.

The protein digestion agent can be present in the protein digestion composition in an amount of between 8 units per sample and 100 units per sample.

Conventional digesting steps in previous expanded microscopy methods have required incubation at elevated temperatures, such as 45-65° C. The inventors surprisingly discovered that the methods and compositions described herein can achieve the necessary digestion and corresponding expansion by incubating at between 35° C. and 40° C., including 37° C., for between 15 min and 48 hours, including between 18 hours and 36 hours or between 21 hours and 30 hours.

The expanding of process blocks 118 and 218 can involve initiating expansion of the ExM monomers using an activation mechanism associated with the ExM monomers. In some cases, the expanding involves incubating the gel at room temperature in pure water for between 1 hours and 12 hours, periodically replacing the water with fresh pure water. In some cases, particularly for ×4 ExM, the expanding is done for 2 hours, exchanging the water for fresh pure water every 30 minutes. For a 4-fold gel, PBS or other physiological buffer can be used instead of water, but for a 12-fold gel, the buffer containing phosphate such as PBS causes shrinkages. Note, those buffers containing phosphate can be used to re-shrinkage for a 12-fold ExM. It can shrink to the pre-expansion size by PBS wash, then can be re-expanded again by water wash. In some cases, particularly for ×12 ExM, the expanding is done for 8 hours, exchanging the water for fresh pure water every 2 hours.

In some cases, the expanding involves water sufficient to cover the entire gel. In some cases, the expanding involves only partially submerging the gel in the pure water.

In some cases, the gels are removed from the molds prior to expanding. In some cases, the gels are shielded from light during the expanding.

The preparing the expanded sample of interest for imaging of process blocks 120 and 220 can involve conventional approaches to preparing a three-dimensional sample for imaging.

In some cases, the preparing of process block 120 and 220 includes post-staining.

In some cases, the preparing of process block 120 and 220 includes removing the expanded gel from water, and mounting the expanded gel within a mounting medium comprising between 20 and 100% glycerol. The mounting medium can further include an anti-fade reagent to suppress photo-bleaching. The anti-fade reagent can include n-propyl gallate, dimethyl sulfoxide, tris(hydroxymethyl)aminomethane, other anti-fade reagents known to those having ordinary skill in the art, or combinations thereof.

In some cases, preparing of process blocks 120 and 220 can include applying pressure and/or compressing the expanded sample of interest to reduce its size in a dimension along which the imaging is intended to proceed. In other words, the sample of interest is compressed in the dimension along the optical axis of the imaging modality. Preparing can also include introducing the expanded gel into a second mold that is dimensioned to stably maintain the gel's position during imaging.

The imaging of process blocks 122 and 222 can include conventional microscopy techniques, though more complicated techniques are contemplated for use with the present disclosure. One of the unexpected results of the present disclosure is achieving reliable expansion of samples, such that conventional microscopy systems can be utilized to observe features that are typically below the diffraction limit of such systems.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1: Mouse Embryo Preparation

The following example is a method for immunostaining embryos, including mouse embryos, in advance of performing an expansion protocol as described in further examples provided below.

### Immunostaining

A) Transfer embryos into a 96-hole plate containing 0.01% PVA/PBS solution.
B) Remove ZP with Acidified Tyrodes Solution for ~30 sec.
C) Fix with 4% PFA/PBS at RT for 20 minutes and wash 3 times with 0.01% PVA/PBS solution.
D) Permeabilize with 0.5% Triton X-100/PBS (Fresh) for 20 min at RT in the dark.
E) Wash 3 times with 3% BSA/PBS solution.
F) Primary antibody/3% BSA/PBS (e.g. 1:100, laminB1 antibody) 4° C. O.N and wash 3 times with 3% BSA/PBS solution.
G) Second antibody/3% BSA/PBS (e.g. 1:100, Alex568-GAM) 4° C. O.N and wash 3 times with 3% BSA/PBS solution.

### Example 2: Crosslinking

The following example applies to all biological samples utilized in the disclosed method.

A) Crosslinking with Glycidyl Methacrylate (GMA)
1. Wash 3 the sample times with 0.01% PVA/PBS solution
2. Incubate sample with 100 mM sodium bicarbonate (pH=8.5, DNase/RNase-free, filtered, Fresh) twice for 15 min.
3. Incubate the sample with 0.01 to 0.5% glycidyl methacrylate (GMA) in 100 mM sodium bicarbonate for 12-15 hours at RT.
4. Wash the sample 5 times with PBS solution. Use a capillary coated with 0.1% PVA/PBS inside the capillary.
   B) Crosslinking with AMT-NHS
1. Contact sample with 0.01-1 mM of AMT-NHS (2,5-dioxopyrrolidin-1-yl 3-(((2,5,9-trimethyl-7-oxo-7H-furo[3,2-g]chromen-3-yl)methyl)thio)propanoate) in PBS or other physiological buffer (e.g., N-2-hydroxyethylpiperazine-N′-2-ethanesulfonic acid (HEPES)) for 10 min to 15 hours at RT.
2. Wash the sample 5 times with PBS solution.

### C) Glyoxal Fixation*

1. Contact sample with 1-10% Glyoxal in PBS or other physiological buffer (e.g., N-2-hydroxyethylpiperazine-N′-2-ethanesulfonic acid (HEPES)) or 1-10% Glyoxal/0.5-10 acetic acid for 10 min to 15 hours at RT.
2. Wash the sample 5 times with PBS solution.

*In some cases, glyoxal is used for fixation (e.g., primary fixation). In some cases, glyoxal is used in crosslinking. In some cases, glyoxal is used in both fixation and crosslinking.

D) any Combinations of A) Through C) and/or with Glutaraldehyde

Crosslinking may be accomplished using a combination of methods. The methods may be used in any order (e.g., GMA crosslinking followed by AMT-NHS crosslinking or AMT-NHS crosslinking followed by GMA crosslinking). For example, the sample may be crosslinked using GMA crosslinking and AMT-NHS crosslinking, using GMA crosslinking and glyoxal fixation, with AMT-NHS crosslinking and glyoxal fixation. In some cases, all three methods (GMA crosslinking, AMT-NHS crosslinking, and glyoxal fixation) may be used.

Any of the combinations described above may also be combined with crosslinking using glutaraldehyde (GA). The glutaraldehyde crosslinking method includes preparing a solution of glutaraldehyde f.c. (final concentration) 0.1~2% of GA in 1× PBS/PHEM and at least one of the crosslinking agents (GMA, AMT-NHS, or glyoxal) at a concentration of the crosslinking agent in the methods described in sections A) though C) above, respectively. Incubate the sample for 1 hour to overnight (13-18 hours) at room temperature with protection from light. In some cases, some tissue or 3D culture samples, such as organoids, may incubate with fresh glutaraldehyde solution (glutaraldehyde f.c. (final concentration) 0.1~2% of GA in 1×PBS/PHEM) at the same concentration in next day and incubate for 15 min at room temperature.

### Example 3: Exemplary 4× Expansion Protocol

1. Cell preparation; Spread any cells or organoids you would like to do expansion on 12 mm circular coverslips (#1.5 thickness) and grow cells at least two over nights. Avoid confluent when you fix cells in next step. Note that you can use different size or shape of coverslips, but 12 mm circular coverslips fits for current silicone molds we have.

2. Primary Fixation: Fixation can be done by regular methods including PFA, MeOH, Aceton, and GA. Here, we show one of our PFA methods as an example. After two over, fix cells by pre-warmed (37° C.) 3% PFA in PHEM buffer for 15 min at 37° C. PBS can be also used but PHEM buffer preserves cellular architecture better than PBS. Some fixation, such as MeOH, causes shrinkages. However, any fixations can be used for mExM.

3. Pre-ExM staining: Regular immunostaining (IF) protocol can be performed. Immunostaining of embryos, as described in Example 1 above, may be performed at this step. Below is summary of another typical cell immunostaining method. Note that post-ExM staining, staining after expansion, is also available.

3-1. After cells are fixed, cells are washed by PHEM or PBS for three times. If you use high concentration of PFA or GA (glutaraldehyde), quenching step by Glycine or Sodium borohydrate, is required here.

3-2. Blocking by BSA, skim milk, or IgG in PBS or PHEM for 15~30 min at room temperature.

3-3. Incubation with primary antibodies in blocking buffer at 37° C. for at least 1 hour or 4° C. for overnight if needs. Multi-color staining allows using multiple primary antibodies.

3-4. Wash cells by blocking buffer for at least three times.

3-5. Incubation with secondary antibodies in blocking buffer at 37° C. for at least 1 hour or 4° C. for overnight if needs. Note that majority of regular dyes, such as Alexa488, Alexa 568, Cy3, and Rhodamine Red-X, can be used.

3-6. Wash cells by PHEM or PBS

3-7. DNA staining by DAPI or other dye if necessary.

Note that if your cells expressing fluorescent proteins such as EGFP, you can skip IF or you can do IF using EGFP antibodies to enhance signals. Our mExM can detect native EGFP or other fluorescence proteins without IF.

If your cells expressing tags can be stain later such SNAP tag or Halo tags. Staining dyes for those tags can be added before next step (pre-expansion) or after expansion (post-expansion).

4. Crosslink: Coverslips are transferred into 24 well dishes. Any combination of crosslinking methods as described in Example 2 (above) may be used to crosslink the sample.

Additionally, the following three methods can be used, alone or in combination with each other and/or with the methods describes in Example 20:

4 (a) AcX method: Add all into one coverslip in 24 well dishes and Incubate cells with Acryloyl-X, SE (AcX), (f.c. 0.01~0.05 mg/ml) on shakers for overnight (13~18 hrs) at room temperature with protection from light (wrapped by foil). Acryloyl-X 10 mg/mL stock solution can be stored in aliquots of 5 μL at −20° C. for a few months if freeze-thaw cycle is avoided. Stock solution is composed of 500 μL of anhydrous DMSO and 5 mg of Acryloyl-X, SE.

4 (b) GA Method: Glutaraldehyde f.c. (final concentration) 0.1~2% of GA in 1× PBS/PHEM. Incubation time is same as above AcX, but we typically replace fresh GA (same concentration) in next day and incubate just 15 min at room temperature.

4 (c) AcX-GA Method: 0.1~2% of GA and 0.01~0.05 mg/ml of AcX in 1× PBS/PHEM. Incubation time is same as above AcX, but we typically replace fresh GA (same concentration) in next day and incubate just 15 min at room temperature.

5. Wash: Cells are washed by PBS for at least three times.

6. Additional Crosslink: Some tissues or 3D cell cultures, such as organoids, are incubated with fresh 0.25% of GA (glutaraldehyde) in PBS for 15 min at room temperature. Recommended to use EM (electron microscopy) grade of GA.

7. Gelling: Three following buffers are used for gelling of ~x4 ExM.

TABLE 1
Buffer 1, 4x ExM Monomer Solution,
aliquots of 1 mL, store −20° C. for few months

| Chemicals | Final Concentration | |
|---|---|---|
| 10x PBS | 1x | |
| 5M NaCl | 2M | |
| Acrylamide | 2.5% | w/v |
| N,N'-Mathylenebisacrylamide | 0.15% | w/v |
| Sodium Acrylate | 8.6% | w/v |
| Double-distilled | Up to 20 | mL |
| Water (milli Q water) | | |
| Total | 20 | mL |

TABLE 2
Buffer 2, APS, aliquots of 500 μL,
store −20° C. for few months

| Chemicals | Amount | |
|---|---|---|
| Ammonium Persulfate | 0.1 | g |
| Double-distilled water | 1 | mL |
| (milli Q water) | | |

TABLE 3
Buffer 3, TEMED 10% w/v,
aliquots of 20 μL, store
−20° C. for few months

| Chemicals | Amount |
|---|---|
| TEMED | 100 μL |
| Double-distilled water | 900 μL |
| (milli Q water) | |

7-1: Prepare ice bucket (with ice), attach silicone molds to glass-slides, and a humid box (pre-warmed couple of hours prior to next step).

7-2: Infusion: washout with 1×PBS few times, then add 200 μl of ×4 ExM monomer solution (buffer 1 above) and incubate for 30 min at 4° C.

7-3: Polymerization: coverslips are placed in the whole of silicone molds and make gel solution (see below) and add 70 μl of gel solution to each sample, then incubate them in a humid box (pre-warmed and keep humidity) at 37° C. for 1 hr.

Silicone molds can be re-used multiple times (>10 times), but require washing out. If the mold has tears, then it must be discarded and cannot be re-used.

TABLE 5
4x ExM Gel Solution

| Chemicals | Amounts | |
|---|---|---|
| Buffer 1 | 475 | μL |
| Double-distilled water | 5 | μL |
| (milli Q water) | | |
| Buffer 2 | 10 | μL |
| Buffer 3 | 10 | μL |

8. Digestion

TABLE 6
Buffer 4a, Digestion Buffer sans ProK, aliquots
of 5~6 μL, store −20° C. for few months

| Chemicals | Amounts | |
|---|---|---|
| Triton X-100 | 1 | mL |
| 50x TAE | 4 | mL |
| Guanidine HCl | 15.29 | g |
| Double-distilled water | Adjust to 200 | mL |
| (milli Q water) | | |

TABLE 7
Buffer 4b, Digestion Buffer sans ProK, aliquots
of 5~6 μL, store −20° C. for few months

| Chemicals | Amounts | |
|---|---|---|
| Triton X-100 | 1 | mL |
| 50x TAE | 4 | mL |
| Guanidine HCl | 15.29 | g |
| Double-distilled water | Adjust to 200 | mL |

(milli Q water)

8. Digestion

8-1: Thaw Buffer 4a or 4b to become room temperature.

8-2: Prepare Digestion Buffer (Buffer 5).

TABLE 8
Buffer 5, Digestion Buffer (make
before using every time-vortex
well and spind own to remove
drops from the top of a tube)

| Chemicals | Amount |
|---|---|
| Buffer 4a or 4b | 210 μL |
| Proteinase K | 1~10 μL |
| (~8 U/μL) | (8.4 μL) |

8-3: Do not physically disturb gel/sample.

8-4: Add 200 μL of Buffer 5 to each gel/sample, then incubate in a prewired humid box at 37° C. for 1~24 hours (can be done overnight).

9. Expansion

9-1: Transfer gels into dishes (you should use bigger than 60 mm dishes). Usually, cells were transferred into gels.

9-2: Add digestion buffer (2-3 ml to each sample) and incubate at room temperature for 30 min.

9-3: Fill a dish half-full width double-distilled water (milli Q water) and incubate at room temperature for 30 min.

9-4: Exchange double-distilled water (milli Q water) and incubate at room temperature for 30 min.

9-5: Exchange double-distilled water (milli Q water) and incubate at room temperature for 30 min.

Total of two hours. Samples should be shielded from light during the expansion steps. Salt makes the gels shrink, so do not add buffer containing salts, such as PBS. Exchanging water may be critical. Gels do not appear to expand well without exchanging even if samples are incubated for the appropriate length of time.

Regular IF staining or staining by dyes can be done in this step. For example, if you would like to visualize DNA, additional DAPI staining can be performed in this step even if you stained DNA pre-expansion.

10. Post-Staining

TABLE 9
Post-ExM DAPI Staining Composition

| Chemicals | Amount |
|---|---|
| DAPI 10 mg/mL | 20 μL |
| Double-distilled water | 20 mL |
| (milli Q water) | |

10-1: Remove water from expanded gel dishes

10-2: You can perform post staining without cutting gels or after cutting gels.

10-3: Add staining solution onto gels, then incubate gels for appropriate duration.

For example, incubate 40~60 min with DAPI.

10-4: Wash with double-distilled water (milli Q water) at least three times.

11. Image Preparation

11-1: Remove water and add 400 μL of mounting media (Buffer 6) into the dish. When preparing Buffer 6, note that n-propyl gallate does not dissolve in water. Before adding 1M Tris and glycerol, n-propyl gallate is dissolved in DMSO. Add a stir bar and mix gently but thoroughly.

TABLE 11
Buffer 6, Mounting media,
store −20° C. for few months

| Chemicals | Amount | |
|---|---|---|
| n-propyl gallate | 0.2 | g |
| DMSO | 1 | mL |
| 1M Tris (pH 9.0) | 10 | mL |
| Glycerol | 90 | mL |

11-1: Remove water and add 400 μL of mounting media (Buffer 6) into the dish. When preparing Buffer 6, note that n-propyl gallate does not dissolve in water. Before adding 1M Tris and glycerol, n-propyl gallate is dissolved in DMSO. Add a stir bar and mix gently but thoroughly.

11-2: Set a silicone mold on slide grass (different size of the mold used for gelling).

11-3: Cut gels to be a similar size to the hole in a mold.

11-4: Transfer gels with mounting media into a hole of mold, then cover it by coverslips bigger than a silicone mold, and slightly push then seal all by small clips at least two opposite edges. Do not clip inside of a hole. Must clip edge of a mold. Having a mold at this step may be important to obtaining stable imaging results. Without the mold, the gels often shift during imaging and those shifted images are difficult to assess.

11-5: Imaging by appropriate light microscopy. We recommend using an oil objective for best results.

12. Image Analysis

12-1: 2D analysis (length, area, distance) is performed by Nikon Element, Metamorph offline, and MatLab program.

12-2: 3D analysis (3D relative positions, volume, 3D distance, 3D length) is performed by Imaris Software.

### Example 4: 12× Expansion Protocol

The protocol proceeds along the same general protocol as the 4× protocol described in Example 3 above. The differences will be described herein. Portions of the 4× protocol described in Example 3 above that are not discussed here are the same in this protocol.

7. Gelling: Three following buffers are used for gelling of ~×12 ExM.

TABLE 12
Buffer 7, 12x ExM Monomer
Solution, aliquots of 1mL, store
−20° C. for 2~3 weeks, recommend
not to use if older than 3 weeks

| | Final | |
|---|---|---|
| Chemicals | Concentration | |
| DMAA | 1.335 | g |
| Sodium Acrylate | 0.32 | g |
| Double-distilled Water | 2.850 | g |
| (milli Q water) | | |
| Total volume | 4.5 | mL |

7. Gelling: Three following buffers are used for gelling of ~×12 ExM.

7-1: Prepare ice bucket (with ice), attach silicone molds to glass-slides, and a humid box (pre-warmed couple of hours prior to next step).

7-2: KPS and TEMED need to be made fresh. Measure 0.018 g of KPS into a 1.7 mL Eppendorf tube.

7-3: Add 2-3 μL of TEMED in another 1.7 mL Eppendorf tube.

7-4: Thaw a frozen aliquot of Buffer 7.

7-5: Wash cells in 1 mL of PBS several times in 24 well dishes, avoiding directly contacting surfaces of coverslips to avoid removal of cells.

7-6: Gel solution-user should read all of steps 7-6a, 7-6b, 7-7, 7-8, and 7-9 before starting this step.

7-6a: Make “KPS solution” by adding 500 μL of double-distilled water in the Eppendorf tube from step 7-2. Vortex well until no particles are visible. KPS solution should be prepared as close as possible to the time when TEMED is added to the gel solution.

7-6b: Prepare Gel Solution, keeping on ice even during mixing. Gel solution includes 360 μL of Buffer 7, 40 μL of KPS solution, and 1.6 μL of TEMED (100%). The timing of this addition is important (see below). KPS solution addition should be done as close as possible to when TEMED is added. KPS should be added before TEMED and when KPS is added, the solution should be mixed well by pipetting and vortexing, and then quick spun down to remove drops from the top of a tube.

7-7: Move samples (coverslips with cells) in to prepared silicone molds (a hole within a mold) and wash with Buffer 7.

7-8: Complete the Gel solution discussed above by adding TEMED, then quickly mixing. After adding TEMED, the solution initiates gelling, so make sure that the samples are ready to receive the Gel solution.

7-9: Immediately add 70~150 μl of gel solution to each sample, then push edges of sample slips down to ensure no bubbles between glass-slide & sample. The amount of gel solution will depend on the size of molds and coverslips.

7-10: Attach a piece of parafilm (paraffin wax film) to the top of the gel solution, excluding bubbles. Exclusion of bubbles is very important, because polymerization does not occur in the presence of oxygen. However, one significant advantage of this process is that the solution does not need to be actively degassed or deoxygenation by flowing an inert gas like nitrogen through the solution.

7-11: Incubate samples in a humid box at room temperature for 2 hours.

9. Expansion

9-1: Remove gel from mold and glass slide by carefully peeling back mold and attaching non-cell side of gel against the inside of a large dish. Usually, cells were transferred into gels.

9-2: Add double-distilled water (milli Q water), ~50% of dish for first time to help with avoiding gel loss, then incubate it for 2 hours at room temperature.

9-3: Gently exchange double-distilled water (milli Q water) with 75~90% of dish, then incubate it for 2 hours at room temperature.

9-4: Gently exchange double-distilled water (milli Q water) with 75~90% of dish, then incubate it for 2 hours at room temperature.

9-5: Gently exchange double-distilled water (milli Q water) with 75~90% of dish, then incubate it overnight at room temperature.

9-6: The following day, gently exchange double-distilled water (milli Q water) with 75~90% of dish.

Samples should be shielded from light during the expansion steps. Salt makes the gels shrink, so do not add buffer containing salts, such as PBS. Exchanging water may be critical. Gels do not appear to expand well without exchanging even if samples are incubated for the appropriate length of time.

12. Reversing Expansion. In the event that you want to go back to the size of pre-expansion to take images of the same cells before and after expansion or additional staining, perform the steps that follow.

12-1: Place gels back into a dish.

12-2: Add ×1 PBS to half-fill the dish, then incubate for 30 minutes.

12-3: Replace the x1 PBS with fresh PBS, then incubate for 30 minutes.

12-4: Repeat until the gels are a desired size. 12-5: Perform staining or imaging with compressed samples.

13. Re-expansion. In the event that you want to re-expand the reversed/compressed samples, perform the steps that follow.

13-1: Place gels back into a dish.

13-2: Add double-distilled water (milli Q water) and replace water until the gels expand to the desired size.

14. Image analysis. Image analysis is performed as described above in “12. Image Analysis” in the above 4× Expansion protocol.

The present invention has been described above with reference to various exemplary configurations. However, those skilled in the art will recognize that changes and modifications may be made to the exemplary configurations without departing from the scope of the present invention. For example, the various operational steps, as well as the components for carrying out the operational steps, may be implemented in alternate ways depending upon the particular application or in consideration of any number of cost functions associated with the operation of the system, e.g., various of the steps may be deleted, modified, or combined with other steps. These and other changes or modifications are intended to be included within the scope of the present invention, as set forth in the following claims.

1. A kit for modified expansion microscopy (mExM), comprising:
a mold comprising a sealing surface configured to form an airtight seal with a sealing material to exclude air bubbles from the mold;
a crosslinking agent configured to covalently bind to both a biological element of a sample and to an expansion gel; and
instructions for use of the mold in an expansion microscopy method, comprising:
a description of an expansion microscopy method that includes at least one method step that involves gelling in the absence of oxygen, and
a written or visual representation of execution of the expansion microscopy method by conducting the at least one method step that involves gelling in the mold by forming an airtight seal against the sealing surface with a sealing material to exclude visible air bubbles from the mold upon sealing.

2. The kit of claim 1, wherein the crosslinking agent comprises glycidyl methacrylate (GMA), (2,5-dioxopyrrolidin-1-yl)3-[(2,5,9-trimethyl-7-oxofuro[3,2-g]chromen-3-yl)methylsulfanyl]propanoate (AMT-NHS), glyoxal, acryloyl-X (AcX), glutaraldehyde (GA), or a combination thereof.

3. The kit of claim 1, wherein the biological element is a nucleotide.

4. The kit of claim 1, wherein the biological element is selected from the group consisting of a protein, organelles, single-stranded DNA, single-stranded RNA, double-stranded RNA, DNA-RNA hybrid, double-stranded DNA, higher order DNA/RNA structures, nuclei, or any combinations thereof.

5. The kit of claim 1, further comprising a protein digestion agent comprising sodium dodecyl sulfate and a buffering component.

6. The kit of claim 1, wherein the mold is configured to receive a biological sample with sufficient volume to undergo at least a 12-fold increase in volume of the biological sample.

7. The kit of claim 1, wherein the mold comprises silicone or gum.

8. The kit of claim 1, wherein the expansion gel comprises expansion monomers; and
wherein the expansion monomers are selected from the group consisting of sodium acrylate (SA), acrylamide (AA), N,N-methylenebisacrylamide (MBAA), or any combinations thereof.

9. The kit of claim 1, wherein the sealing material is a wax material, a film, a nylon material, a silicone, a flexible hydrophobic material, or any combination thereof.

10. (canceled)

11. A method of modified expansion microscopy (mExM), the method comprising:
a) fixing a biological sample having a biological element to an inert surface, optionally locating the inert surface within a mold comprising a surface configured to form an airtight seal;
b) exposing the biological sample to a crosslinking agent to produce a covalent bond between the crosslinking agent and the biological element;
c) exposing the biological sample to a plurality of expansion monomers and polymerizing at least a portion of the expansion monomers to form covalent bonds between the crosslinking agent and the expansion monomers to form polymerized expansion monomers,
d) optionally introducing the inert surface into the mold;
e) introducing a gelling solution into the mold including the inert surface and the biological sample of interest;
f) subsequent to step e), sealing the mold with a sealing material to exclude air bubbles from the mold;
g) gelling the polymerized expansion monomers to form a gelled biological sample, the gelling proceeding without active deoxygenation;
h) digesting the gelled biological sample by exposing the gelled biological sample to a digestion composition;
i) expanding the gelled biological sample to form an expanded biological sample; and
j) preparing the expanded biological sample for imaging.

12. The method of claim 11, wherein the airtight seal is formed with manual force.

13. The method of claim 11, wherein the crosslinking of step b) uses a crosslinking agent comprising glycidyl methacrylate (GMA), (2,5-dioxopyrrolidin-1-yl)3-[(2,5,9-trimethyl-7-oxofuro[3,2-g]chromen-3-yl)methylsulfanyl]propanoate (AMT-NHS), glyoxal, acryloyl-X (AcX), glutaraldehyde (GA), or a combination thereof.

14. The method of claim 11, wherein the crosslinking of step b) involves two different crosslinking steps.

15. The method of claim 14, wherein the two different crosslinking steps include a first crosslinking step and a second crosslinking step, the first crosslinking step including crosslinking for a length of time of between 10 minutes and 24 hours, the second crosslinking step including crosslinking with glutaraldehyde for a length of time of between 5 minutes and 20 hours.

16. The method of claim 11, the method further comprising an additional crosslinking step subsequent to the crosslinking of step b), the additional crosslinking step comprising incubation with glutaraldehyde for a predetermined length of time.

17. The method of claim 11, wherein the plurality of expansion monomers comprises N,N′-methylenebisacrylamide, N,N′-dimethylacrylamide, or a combination thereof.

18. (canceled)

19. (canceled)

20. The method of claim 11, wherein the contents of the mold are removed prior to the expanding of step i).

21. The method of claim 11, wherein the preparing of step j) includes applying pressure to the expanded biological sample to reduce its size in a dimension along which the imaging is intended to proceed.

22. The method of claim 11, the method further comprising imaging the expanded biological sample subsequent to step j).

23. (canceled)

24. (canceled)

25. (canceled)

26. (canceled)

27. A method of modified expanded microscopy (mExM), the method comprising:
a) fixating a sample of interest to an inert surface, the inert surface located within a mold or the inert surface and the mold adapted such that the inert surface is receivable within the mold;
b) crosslinking biological elements of the sample of interest, the crosslinking comprising glycidyl methacrylate (GMA), (2,5-dioxopyrrolidin-1-yl)3-[(2,5,9-trimethyl-7-oxofuro[3,2-g]chromen-3-yl)methylsulfanyl]propanoate (AMT-NHS), glyoxal, acryloyl-X (AcX), glutaraldehyde (GA), or a combination thereof;
c) infusing the sample of interest with a plurality of expansion microscopy (ExM) monomers and polymerizing at least a portion of the ExM monomers;
d) optionally introducing the inert surface into the mold;
e) introducing a gelling solution into the mold including the inert surface and the sample of interest;
f) optionally providing an airtight seal to the mold, the airtight seal excluding air bubbles from the mold, the airtight seal capable of being formed with manual force;
g) gelling the plurality of polymerized ExM monomers;
h) digesting proteins within the sample of interest, the digesting comprising use of a protein digestion agent and a protein digestion buffer, the protein digestion buffer comprising sodium dodecyl sulfate and a buffering component;
i) expanding the sample of interest by initiating expansion of the gelled, polymerized ExM monomers; and
j) preparing the expanded sample of interest for imaging.
